(19) United States
(12) Patent Application Publication
(10) Pub. No.: US 2026/0232562 A1
(43) Pub. Date: Aug. 13, 2026

(54) COSMETIC COMPOSITION COMPRISING A RECOMBINANT BACTERIAL COLLAGEN-LIKE PROTEIN (CLP) AND USES THEREOF

(71) Applicant: Evonik Operations GmbH, Essen (DE)

(72) Inventors: Corinna Marie Reisinger-Becker, Hanau (DE); Yun Mu, Moseley, VA (US); Anna Jacobi, Seligenstadt (DE); Steffen Osswald, Nidderau (DE)

(73) Assignee: Evonik Operations GmbH, Essen (DE)

(21) Appl. No.: 19/153,538

(22) PCT Filed: Feb. 1, 2024
(86) PCT No.: PCT/EP2024/052467

§ 371 Date: Aug. 4, 2025

(30) Foreign Application Priority Data
Feb. 9, 2023 (EP) 23155702.6

(51) Int. Cl. A61K 8/65; A61Q 5/06; A61Q 19/08

(52) U.S. Cl. CPC A61K 8/65; A61Q 5/06; A61Q 19/08; A61K 2800/85; A61K 2800/92

(57) ABSTRACT

The present invention relates to cosmetic compositions including a recombinant collagen-like protein (CLP). A cosmetic composition may include a recombinant bacterial collagen-like protein having an amino acid sequence at least 60% identical to SEQ ID NO:1 with deletion of at least 38 amino acids at the N-terminus for anti-aging applications.

The present invention relates to a cosmetic composition comprising a recombinant collagen-like protein (CLP) both as active and functional ingredient and uses thereof.

Collagen is a longstanding player in anti-aging cosmetics and is mostly derived from animal sources. As such, it does not meet today's increasingly stringent standards of conscious beauty with growing demand for vegan, clean and green ingredients.

Collagen in cosmetics serves a broad range of applications from being a topically applied skin care active to serving as a functional in hair styling products. Different applications require tailored material properties and exploit the various material features of the collagen from animal-sources.

Collagen from animal sources forms a stable triple-helical structure. However, when the respective mammalian collagen sequence is expressed in a lower order organism such as a bacteria or a yeast, the material does not exhibit a critical degree of post-translational hydroxylation to stabilize the triple-helical structure. Therefore, recombinantly produced collagen-like proteins by fermentation are generally available only as single strand and do not fold into a stable triple helix.

In WO2020/205848A1 for example topical formulations of recombinant collagen are disclosed and U.S. Pat. No. 11,514,609B2 describes recombinant collagen and elastin molecules and uses thereof. However, those products are in a monomeric form and do not form a stable tripe helical structure similar to natural collagen.

It was therefore a subject of the present invention to provide a vegan collagen alternative for cosmetic applications, which also can adopt a triple-helical structure.

The present invention therefore relates to a cosmetic composition comprising a recombinant bacterial collagen-like protein. Preferably, the recombinant bacterial CLP is a bacterial collagen-like protein from Streptococcus pyogenes.

The bacterial collagen-like protein mimics the animal-derived material in all aspects, also with regard to the triple helical structure. In that sense, it is ahead of the current offerings in the area of collagen-like proteins by fermentation. The intrinsic structural properties of the bacterial collagen depending on the specific sequence allows the material to adopt a stable, triple-helical structure mimicking natural, human collagen, also when expressed in a host organism. In addition, the material is also accessible as single-stranded product showcasing the superior versatility.

In addition, the use of bacterial collagen renders it unnecessary to introduce specific additional enzymes into the host to enable critical post-translational hydroxylation during the fermentation process as these modifications are not needed to stabilize the triple helix due to the structural uniqueness of bacterial collagen.

The current invention of using bacterial collagen-like protein in cosmetics overcomes the limitation, that vegan collagen by fermentation is generally only available as single-stranded molecule for such applications but not as the stable triple-helical form. Therefore, the application of bacterial collagen-like protein in cosmetics enables the versatile use of vegan collagen in cosmetics-either as triple helix or single strand.

Therefore, the material is suitable for a broad range of personal care applications as active and functional ingredient in topical skin care all the way towards hair & lash styling products.

### Structure of Recombinant Bacterial CLPs

Collagen-like proteins (CLPs) of bacterial origin (the most industrially relevant being the product of Streptococcus pyogenes) have considerably interesting mechanical properties, similar to those of higher eukaryotes' collagen proteins, without needing the complex maturing steps required for the eukaryotic counterparts. CLPs present a common structure: two alpha helixes, stabilizing each other, constitute a “V domain”, which is followed by a rod-like, structural collagen domain (CL). After the collagen domain, typically a membrane anchor (GPI-like) is present at the C-terminal end of the protein.

As described in various publications (Lukomski et al. 2002 (J. Biol. Chem., 277:27312-27318, 2002), Brodsky et al. 2009 (Protein Sci., 18:1241-1251, 2009)) the current understanding of this process is that the V-domain is required for folding three Scl2 protein monomers into one triple helical structure in vitro (Lukomski et al. reveals in vivo folding without V-domain).

Even though the V-domain might have a positive effect on this process it was found that it's not the sole factor for folding the protein. It could be shown that a proper folding also takes place in absence of the V-domain. The main factors identified are concentration of the Scl2 monomer, temperature, time, pH-value und salt concentration.

Since the V-domain was thought to be crucial for production of triple helical Scl2 it was never considered to remove this sequence leading to the following challenges:
The V-domain makes up for approximately one third of the whole sequence and hinders the protein to be transported out of the Pichia pastoris host. This requires a complex downstream process containing cell lysis to remove the target protein from the cell.
The V-domain itself has pathogenic properties and needs to be removed during the purification process. This is done by a protease digest. Usage of a protease is quite costly, and it needs to be removed during downstream as well.

The following summary shows the process steps required for a product purification using a Scl2 construct with V-domain attached:
Cell separation (Centrifugation)
Cell lysis (Pressure homogenizer)
V-domain removal (Protease digest)
Removal of cell debris (pH-shift, centrifugation)
Purification (Solvent precipitation)
Washing (TFF)
Further purification (IEX)
   Such a process is disclosed in Peng et al. (Appl. Microbiol. Biotechnol., 98:1807-1815, 2014) for example.

### Production of Recombinant Bacterial CLPs

The collagen-like proteins (CLPs) can be produced in the methylotrophic yeast Pichia pastoris or other hosts. The key features of such process, compared to the current process known from the prior art: 1) proteins are secreted in culture supernatant, allowing to reach a high titer (>5 g/L), in a low-cost medium; 2) proteins are easily purified from the supernatant, since no complex component is present in the cultivation medium.

Surprisingly, the purified product from supernatants of Pichia pastoris cultivation secreting Scl2p, showed an unexpected profile, compatible with mature collagen-like sequences. Further analysis showed how intracellular enzymes, most likely the processing protease Kex2p, are capable to remove the V domain protein sequence without any need of an additional protease step. In addition, in order to modify a cleavage site present in the final product, resulting in significant accumulation of degradation products, the protein sequence has been mutated to engineer such cleavage site and abolish degradation. Unexpectedly, the most efficient performance was obtained when an apolar amino acid (valine, in the wild-type sequence) was mutated to a polar amino acid (glutamine).

It was a surprising finding that the recombinant CLP could after secretion in the culture supernatant be correctly folded in absence of the V-domain during storage/freezing of the cell pellet before lysis. With the present invention a new downstream process is disclosed, including production of the Scl2 protein without V-domain and the intentional integration of a folding step for the CL domain.

The method for producing a recombinant collagen-like protein (CLP) comprises the following steps:
a) fermentation of a host cell, expressing a CLP with an amino acid sequence that is at least 60% identical to the amino acid sequence of SEQ ID NO:1, in a medium, wherein the amino acid sequence comprises a deletion of at least 38 amino acids at the N-terminus of the amino acid sequence of SEQ ID NO:1,
b) accumulation of the CLP in the medium, wherein a fermentation broth is obtained,
c) separating the host cells from the fermentation broth to obtain a supernatant,
d) incubating the supernatant of fermentation broth of step c) for at least 1 h at not more than 25° C. for folding of the CLP,
e) optionally purification of the CLP by at least one of the following: solvent precipitation, tangential flow filtration (TFF), ion exchange chromatography, reversed-phase chromatography.

The host cell is preferably selected from bacterial, yeast of plant cells. It is preferred to use bacterial or yeast cells.

In a preferred embodiment, the host cell is a microorganism of the species P. pastoris, E. coli, P. putida or C. glutamicum comprising any of the polypeptides according to the present invention. In a preferred embodiment, the microorganism is a yeast of the genus P. pastoris or a bacterial cell, preferably E. coli, Corynebacterium or Brevibacterium.

The microorganism may be a microorganism in which the nucleotide sequence is present in overexpressed form.

The microorganism may be characterized in that the microorganism has the capability of producing and secreting a fine chemical. The fine chemical being preferably a collagen-like protein.

Overexpression is taken to mean, generally, an increase in the intracellular concentration or activity of a ribonucleic acid, a protein (polypeptide) or an enzyme, compared with the starting strain (parent strain) or wild-type strain, if this is the starting strain. A starting strain (parent strain) is taken to mean the strain on which the measure leading to the overexpression was carried out.

In the overexpression, the methods of recombinant overexpression are preferred. These include all methods in which a microorganism is produced using a DNA molecule provided in vitro. Such DNA molecules comprise, for example, promoters, expression cassettes, genes, alleles, encoding regions etc. These are converted into the desired microorganism by methods of transformation, conjugation, transduction or like methods.

The extent of the expression or overexpression can be established by measuring the amount of the mRNA transcribed by the gene, by determining the amount of the polypeptide, and by determining the enzyme activity.

It was a surprising finding that truncated variants of the collagen-like protein, including variants with a truncated V-domain or without any V-domain lead to increased production of collagen-like protein and secretion into the fermentation medium. It was further surprising that the truncated variants could be correctly folded in absence of the V-domain.

In a preferred embodiment of the present invention, the optionally purified CLP is folded in an additional process step (step d)). Said folding of CLP in step d) is performed at a temperature between −80° C. and 25° C., preferably between 0° C. and 20° C. In a preferred configuration folding is performed in presence of glycerin or salts.

In another preferred embodiment, folding of CLP in step d) is performed for a time between 1 h and 48 h, preferably between 1 h and 24 h.

In another preferred embodiment, folding of CLP in step d) is performed with a concentration of CLP of at least 1 mg/ml, preferably at least 4 mg/ml.

### Protein Sequences

SEQ ID NO:1 Streptococcus pyogenes Collagen-like protein (CLP), full length protein
SEQ ID NO:2 Streptococcus pyogenes CLP, truncation 3
SEQ ID NO:3 Streptococcus pyogenes CLP, truncation 5
SEQ ID NO:4 Streptococcus pyogenes CLP, no V-domain
SEQ ID NO:5 Streptococcus pyogenes CLP, truncation 5 (AGPR mutant)
SEQ ID NO:6 Streptococcus pyogenes CLP, truncation 5 (QGPR mutant)
SEQ ID NO:7 Streptococcus pyogenes CLP, truncation 5 (VGPA mutant)
SEQ ID NO:8 Streptococcus pyogenes CLP, truncation 5 (SGPR mutant)
SEQ ID NO:9 Streptococcus pyogenes CLP, truncation 5 (VGPK mutant)

In a preferred embodiment, the CLP has an amino acid sequence which is at least 60% identical to the amino acid sequence of SEQ ID NO:1 and the amino acid sequence comprises a deletion of at least 38 amino acids at the N-terminus of the amino acid sequence of SEQ ID NO:1.

It is preferred, when the amino acid sequence comprises a deletion of between 38 and 90 amino acids at the N-terminus of the amino acid sequence of SEQ ID NO:1. This includes a complete deletion of the N-terminal V-domain (comprising 74 amino acids) and different truncations of the V-domain of at least 38 amino acids.

The invention correspondingly also relates to polypeptide variants of SEQ ID NO:2 to 9, which contain one or more insertion(s) or deletion(s). Preferably, the polypeptide contains a maximum of 5, a maximum of 4, a maximum of 3, or a maximum of 2, insertions or deletions of amino acids. In a preferred embodiment, the amino acid sequence that is at least 60%, identical to the amino acid sequence of SEQ ID NO:2, SEQ ID NO:3, SEQ ID NO:4, SEQ ID NO:5, SEQ ID NO:6, SEQ ID NO: 7, SEQ ID NO:8 or SEQ ID NO:9.

In a preferred configuration the amino acid sequence is at least 90%, 92%, 94%, 96%, 97%, 98%, 99% or 100%, preferably 97%, particularly preferably 98%, very particularly preferably 99%, and extremely preferably 100%, identical to the amino acid sequence of SEQ ID NO:2, SEQ ID NO:3, SEQ ID NO: 4, SEQ ID NO:5, SEQ ID NO:6, SEQ ID NO:7, SEQ ID NO:8 or SEQ ID NO:9.

### Cosmetic Composition and Formulations

The cosmetic composition according to the present invention provides one or more benefits for hair care and skin care, including, but not limited to improvement in many signs of skin aging, particularly particular in the face and on the neck, such as lines, wrinkles, crepiness, and sagging, the benefits conferred by one or more of increasing firmness, increasing elasticity, improving hydration, and reducing the presence superficial adipose tissue.

The cosmetic composition according to the present invention preferably comprises between 0.001% and 30% w/w, between 0.01% and 15% w/w or between 0.1% and 10% w/w of the CLP. The cosmetic composition preferably further comprises at least one additional ingredient selected from the group consisting of emollients, emulsifiers, thickeners/viscosity regulators/stabilizers, UV light protection filters, antioxidants, hydrotropes, solids and fillers, film formers, anticaking agents, pearlescence additives, deodorant and antiperspirant active ingredients, insect repellents, self-tanning agents, preservatives, conditioning agents, perfumes, dyes, odour absorbers, superfatting agents, carrier materials, additional skin actives and solvents. Substances which can be used as exemplary representatives of the individual groups are known to those skilled in the art and can be found for example in German patent application DE 10 2008 001788.

As regards further optional components and the amounts used of these components, reference is made expressly to the relevant handbooks known to those skilled in the art, for example K. Schrader, “Grundlagen und Rezepturen der Kosmetika [Cosmetics-fundamentals and formulations]”, 2nd edition, pages 329 to 341, Hüthig Buch Verlag Heidelberg.

In a further aspect, the present invention provides a topical cosmetic formulation comprising a recombinant bacterial collagen-like protein. Said topical cosmetic formulation may comprise at least one cosmetically acceptable carrier.

Delivering active substances, such as CLPs to and through the skin involves the complex interplay between the active substance, the type of carrier system (‘vehicle’), the choice of excipients, skin type and location, and skin condition. The cosmetically acceptable carrier is defined as the substance that carries the chosen active substance into contact with and through the skin at an appropriate level to provide a cosmetical effect. At the same time, said carrier must not affect skin health.

The challenge to topical drug delivery is the transport across the stratum corneum (SC). To overcome this barrier, a carrier must: maintain the solubility and stability of the active substance; release the active substance, depositing it on the skin with even distribution; enable penetration into and permeation through the SC skin barrier; facilitate partitioning from the SC into and diffusion through the viable epidermis; sustain the active substance at the target site for a sufficient duration to provide a cosmetic effect; and limit systemic absorption. Furthermore, a carrier should be soothing and comfortable, spread easily and be aesthetically pleasant.

Topical carriers can be classified based on their physical state, including semisolids (e.g., ointments, creams, gels) and liquids (e.g., lotions, solutions, foams, sprays). Lotions, gels and solutions are monophasic, ointments and creams are biphasic, whilst foams are triphasic. The cosmetically acceptable carrier is such that the composition can be prepared as decorative cosmetic, skin care, body care a wash-off or leave-on hair care composition.

According to one aspect the cosmetically acceptable carrier comprises water. According to another preferred aspect, the carrier additionally comprises a surfactant. The at least one cosmetically acceptable carrier may thus be an aqueous carrier in a single phase, biphase or emulsion.

In accordance with the above, the present invention is also directed to the use of a recombinant bacterial collagen-like protein in cosmetic applications. Said recombinant bacterial collagen-like protein preferably comprises one of the protein sequences of SEQ ID 1 to 9 as defined above.

As an alternative embodiment, the recombinant bacterial collagen-like protein may be formulated for oral consumption as nutricosmetic supplements. The term “nutricosmetics” as used herein is to be understood as products and ingredients that act as nutritional supplements to care skin, nails, and hair. In some embodiments, the recombinant bacterial collagen-like protein can be formulated for oral consumption as a food product or a food ingredient. Accordingly, the present invention provides an oral formulation comprising a recombinant bacterial collagen-like protein.

Said oral formulation may further comprise at least one ingredient selected from carriers, preservatives, and/or additional edible ingredients. Thus, for example, the composition may include vitamins (e.g. vitamin A, vitamin B, vitamin C, vitamin D, vitamin E, etc.), minerals (e.g., calcium, zinc, copper, manganese, chromium, molundenum, boron, etc), sugar (e.g., cellulose, dextrose, maltose, etc.), and/or natural extracts (e.g., herb, ginseng, echinacea, green tea, glucosamine, omega-3, lutein, folic acid, liver oil, fish oil, coffee extracts, etc.). Formulations suitable for consumption by an individual (e.g., a human) include, without limitation, ready-to-mix powders, ready-to-drink beverages, functional shots, supplement tablets and capsules.

In accordance with the above, the present invention is also directed to the use of a recombinant bacterial collagen-like protein as a nutricosmetic. Said recombinant bacterial collagen-like protein preferably comprises one the protein sequences of SEQ ID 1 to 9 as defined above.

Finally, the present invention provides a non-therapeutic method of improving the appearance of the skin, the hair, and/or the nails of a subject, the method comprising administering to the subject a cosmetic composition according to the present invention. The cosmetic composition may be administered topically or orally.

### EXAMPLES

### A) Production of Bacterial Collagen-Like Protein (CLP)

The collagen-like protein was produced in the yeast host cell Pichia pastoris by fermentation. To produce Scl2 from Streptococcus pyogenes in Pichia pastoris, the sequence of the collagen-like protein (full-length protein and truncated variants and no-V-domain variant), has been codon optimized using different algorithms, and cloned in a secretion vector for Pichia pastoris. The sequences used are summarized in SEQ ID NO:1 to SEQ ID NO:9. For each of the specific sequences, a vector was transformed in Pichia pastoris following standard protocol and a standard expression protocol in fed-batch mode was applied (Damasceno, L. M., Huang, C J. & Batt, C. A. Protein secretion in Pichia pastoris and advances in protein production. Appl Microbiol Biotechnol 93, 31-39 (2012)). The collagen domain of the Scl2p protein was detected via HPLC analysis in the supernatant of cell culture. Upon fermentation, supernatant has been separated from biomass via centrifugation (12000 g, 5 mins at room temperature).

The collagen domain of the Scl2p protein based on the sequences SEQ ID NO:1 to SEQ ID NO:9 could be produced under similar conditions using either E. coli, B. choshinensis or C. glutamicum. In case of a production in yeast or C. glutamicum, the collagen domain is secreted by the cell. No cell lysis is needed as an initial purification step in this approach. In case of a production in E. coli a cell lysis is mandatory to remove the collagen domain from the cell.

The full-length collagen-like protein, a truncated variant (truncation 3) and the no-V-domain variant (based on the gene scl2 from Streptococcus pyogenes) were also expressed in Brevibacillus choshinensis. Therefore, the corresponding DNA sequences were cloned into a suitable secretion vector for B. choshinensis. Transformation of B. choshinensis with the new constructed plasmids was done according to Mizukami et al. 2010 (Curr Pharm Biotechnol 2010, 13:151-258).

The B. choshinensis strains were analyzed for their ability to produce the different collagen-like proteins in batch cultivations at 33° C. and pH 7 using the DASGIP® parallel bioreactor system from Eppendorf (Hamburg, Germany). The fermentation was performed using 1 L reactors. The production medium (TM medium, Biomed Res Int 2017, 2017:5479762) contained 10 g/L glucose. Upon fermentation, supernatant has been separated from biomass by centrifugation and was used for SDS PAGE analysis. For all three variants, collagen domain of the Scl2p protein was produced. The full-length collagen-like protein and the no-V-domain variant (based on the gene scl2 from Streptococcus pyogenes) were also expressed in Corynebacterium glutamicum. Therefore, the corresponding DNA sequences were cloned together with an upstream located signal peptide for protein secretion into a shuttle vector for C. glutamicum (Biotechnology Techniques 1999, 13:437-441.). The C. glutamicum strain ATCC 13032 was transformed with the new constructed plasmids by means of electroporation as described by Ruan et al. (Biotechnology Letters 2015, 37:2445-2452).

The C. glutamicum strains were analyzed for their ability to produce the different collagen proteins in fed-batch cultivations at 30° C. and pH 7 using the DASGIP® parallel bioreactor system from Eppendorf (Hamburg, Germany). The fermentation was performed using 1 L reactors. The production medium contained 20 g/L glucose in the batch phase and the fed-batch phase was run with a glucose feed of 4 g/L*h. Upon fermentation, supernatant has been separated from biomass by centrifugation and was used for HPLC analysis. For both variants, collagen domain of the Scl2p protein was produced. For the truncated variant of the collagen-like protein, titer was higher as for the full-length variant.

The process steps are summarized below:
1. Production of collagen-like protein in yeast, E. coli or Corynebacterium
2. Cell lysis (only for E. coli)
3. Cell separation (Filtration or centrifugation)
4. Folding of the CL single strand to form a triple helical structure
5. Further purification by solvent precipitation and ultrafiltration
6. Freeze dry of the purified CL protein
   B) Cosmetic Effects and Formulations with Bacterial Collagen-Like Protein (CLP)

A series of in vitro experiments were conducted to assess the effects of bacterial collagen-like protein (CLP) on normal human dermal fibroblasts.

### Example 1: Bacterial Collagen-Like Protein (CLP) Stimulates Fibroblast Production of the HAS1 Hyaluronan Synthase 1 Gene

Normal human dermal fibroblasts were evaluated for hyaluronan synthase 1 expression (HAS1). The expression of HAS1 was analyzed using RT-qPCR method on total RNA extracted from the cell. Fibroblasts were incubated with an exemplary bacterial collagen-like protein (CLP) of SEQ ID NO: 3 at various concentrations for 24 h. These fibroblasts expressed higher levels of HAS1 than the untreated control or fibroblasts incubated with two different collagen benchmark materials as shown in FIG. 1. CLP stimulated HAS1 expression with up to 452% of the control when tested at 1 mg/ml.

### Example 2: Bacterial Collagen-Like Protein (CLP) Stimulates Procollagen I Release by Fibroblasts

Normal human dermal fibroblasts were evaluated for procollagen I release. Fibroblasts were incubated with an exemplary bacterial collagen-like protein (CLP) of SEQ ID NO 3 at various concentrations for 72 h. At the end of the incubation time, culture supernatants were collected and analyzed by enzyme-linked immunosorbent assay (ELISA) for Procollagen Type I C-peptide (PIP). These fibroblasts released higher levels of procollagen I than the untreated control or fibroblasts incubated with two different collagen benchmark materials as shown in FIG. 2. CLP stimulated procollagen I release by fibroblasts up to 219% at 10 mg/ml (170% at 3 mg/ml and 154% at 1 mg/mL) vs. the untreated control.

Bacterial collagen-like protein (CLP) can be applied in various product forms and formulations. Solubility of bacterial collagen-like protein (CLP) in triple-helical form is >50 g/L in water. In the single-stranded form, its water solubility is increased. Exemplary formulations containing bacterial collagen-like protein (CLP) are listed.

### Example 3: Collagen Cream

Preparation protocol: Heat phase A and B separately to 70-75° C. Add phase A to phase B with stirring. (If phase A has to be charged into the vessel first, phase B must be added without stirring.) Homogenize. Cool with gentle stirring.

TABLE 1
Composition of collagen cream

| Phase | Ingredients | % w/w |
|---|---|---|
| A | TEGO ® Care 450 (Polyglyceryl- | 3.0 |
| | 3 Methylglucose Distearate) | |
| | TEGIN ® M Pellets (Glyceryl Stearate) | 2.0 |
| | TEGO ® Alkanol 18 (Stearyl Alcohol) | 1.0 |
| | TEGOSOFT ® CT (Caprylic/Capric Triglyceride) | 9.5 |
| | TEGOSOFT ® TN (C12-15 Alkyl Benzoate) | 9.5 |
| B | Bacterial collagen-like protein (CLP) | 0.2 |
| | Glycerin | 3.0 |
| | Water | 71.8 |
| Z | Preservative, Perfume | q.s. |

### Example 4: Deep Wrinkle Smoothening Cream with Collagen

Preparation protocol: Heat phase A and B separately to approximately 80° C. Add phase A to phase B with stirring. (If phase A has to be charged into the vessel first, phase B must be added without stirring). Homogenize. Cool with gentle stirring to approximately 60° C. and add phase C. Homogenize for a short time. Cool with gentle stirring and add phase D below 40° C.

TABLE 2
Composition of Deep Wrinkle smoothening cream with Collagen

| Phase | Ingredients | % w/w |
|---|---|---|
| A | TEGO ® Care LTP (Sorbitan Laurate; Polyglyceryl-4 Laurate; Dilauryl Citrate) | 2.0 |
| | TEGIN ® M Pellets (Glyceryl Stearate) | 3.5 |
| | TEGO ® Alkanol 18 (Stearyl Alcohol) | 3.5 |
| | TEGOSOFT ® CT (Caprylic/Capric Triglyceride) | 8.0 |
| | TEGOSOFT ® OP (Ethylhexyl Palmitate) | 8.0 |
| B | SKINMIMICS ® | 5.0 |
| | (Ceteareth-25; Glycerin; Cetyl Alcohol; Behenic Acid; Cholesterol; Ceramide | |
| | NP; Ceramide NS; Ceramide EOS; Ceramide AP; Caprooyl | |
| | Bacterial collagen-like protein (CLP) | 0.2 |
| | Glycerin | 3.0 |
| | Water | 66.3 |
| C | TEGO ® Carbomer 134 (Carbomer) | 0.1 |
| | TEGOSOFT ® OP (Ethylhexyl Palmitate) | 0.4 |
| D | Sodium Hydroxide (10% in water) | q.s. |
| Z | Preservative, Perfume | q.s. |

### Example 5: Lift Effect Face Care Collagen Lotion

Preparation protocol: Heat phase A to 70° C. and phase B to approximately 30° C. Add phase B to phase A without stirring. Homogenize. Cool slowly while stirring.

TABLE 3
Composition of Lift Effect Face Care Collagen lotion

| Phase | Ingredients | % w/w |
|---|---|---|
| A | TEGO ® Care PBS 6 MB (Polyglyceryl-6 Stearate (and) Polyglyceryl-6 | 3.00 |
| | Behenate) | |
| | TEGIN ® M Pellets (Glyceryl Stearate) | 1.00 |
| | TEGO ® Alkanol 1618 (Cetearyl Alcohol) | 1.00 |
| | TEGOSOFT ® DEC (Diethylhexyl Carbonate) | 9.00 |
| | TEGOSOFT ® CT (Carpylic/Capric Triglyceride) | 9.00 |
| | TEGOSOFT ® OER (Oleyl Erucate) | 2.00 |
| B | Bacterial collagen-like protein (CLP) | 0.20 |
| | HyaCare ® 50 (Hydrolyzed Hyaluronic Acid) | 0.10 |
| | Glycerin | 2.00 |
| | Water | 72.70 |
| Z | Preservative, Perfume | q.s. |

### Example 6: Anti-Gravity Face Care Mousse

Preparation protocol: Heat phase A up to 40° C. Add phase A to phase B with stirring. (If phase A has to be charged into the vessel first, phase B must be added without stirring.) Homogenize. Add phase C and homogenize for a short time. Add phase D and stir well. Add phase E and stir well. Mix with propellants (Propan, Butan, Isobutan) at the ratio emulsion 90: propellant 10.

TABLE 4
Composition of Anti-gravity Face Care Mousse

| Phase | Ingredients | % w/w |
|---|---|---|
| A | ABIL ® Care XL 80 (Bis-PEG/PPG-20/5 PEG/PPG-20/5 Dimethicone; Methoxy | 2.50 |
| | PEG/PPG-25/4 Dimethicone; Caprylic/Capric Triglyceride) | |
| | Belsil DM 5, Wacker (Dimethicone) | 5.00 |
| | TEGOSOFT ® DEC (Diethylhexyl Carbonate) | 4.00 |
| | TEGOSOFT ® CT (Caprylic/Capric Triglyceride) | 3.00 |
| | TEGOSOFT ® CR (Cetyl Ricinoleate) | 1.00 |
| B | Bacterial collagen-like protein (CLP) | 0.20 |
| | SK-INFLUX ® V MB(Ceramide NP; Ceramide AP; Ceramide EOP; | 1.00 |
| | Phytosphingosine; Sodium Lauroyl Lactylate; Carbomer; Xanthan Gum) | |
| | TEGO ® Cosmo C 100 (Creatine) | 0.50 |
| | Panthenol | 0.20 |
| | Allantoin | 0.20 |
| | Water | 76.70 |
| C | TEGOSOFT ® OS (Ethylhexyl Stearate) | 1.60 |
| | TEGO ® Carbomer 141 (Carbomer) | 0.15 |
| | TEGO ® Carbomer 140 (Carbomer) | 0.15 |
| | Keltrol CG-SFT (Xanthan Gum) | 0.10 |
| D | Sodium Hydroxide (10% in water) | q.s. |
| E | TEGO ® Betain 810 (Capryl/Capramidopropyl Betaine) | 1.00 |
| | REWOTERIC ® AM C (Sodium Cocoamphoacetate) | 1.00 |
| | Phenoxyethanol; Ethylhexylglycerin (Euxyl PE 9010, Schülke & Mayr GmbH) | 0.70 |
| Z | Perfume | q.s. |

### Example 7: Boost Hair & Scalp Wellness with Mild Cleansing

Preparation protocol: Heat phase A and B to 75-80° C. Add phase B slowly step by step while stirring. Cool with gentle stirring. Add phase C below 40° C.

TABLE 5
Composition of Boost hair & scalp wellness with mild cleansing

| Phase | Ingredients | % w/w |
|---|---|---|
| A | TEGO ® Solve 61 (Polyglyceryl-6 Caprylate; Polyglyceryl-3 Cocoate; | 2.0 |
| | Polyglyceryl-4 Caprate; Polyglyceryl-6 Ricinoleate) | |
| | Rheance One (Glycolipid) | 2.0 |
| | SPHINGONY ® (Sphinganine) | 0.1 |
| B | Glycerin | 3.0 |
| | Bacterial collagen-like protein (CLP) | 0.005 |
| | Water | 88.9 |
| C | dermosoft ® 1388 eco (Glycerin; Aqua; Sodium Levulinate; Sodium Anisate) | 4.0 |

### BRIEF DESCRIPTION OF THE FIGURES

FIG. 1 illustrates the effect on gene expression of hyaluronan synthase 1 (HAS1) in fibroblasts treated with an exemplary bacterial collagen-like protein (CLP) (C) and benchmark materials A and B.

FIG. 2 illustrates the effect on procollagen I release by fibroblasts treated with an exemplary bacterial collagen-like protein (CLP) (C) and benchmark materials A and B; ns: >0.05 not significant, *: 0.01 to 0.05 significant, **: 0.001 to 0.01 very significant; ***: <0.001 extremely significant, nc: not calculable.

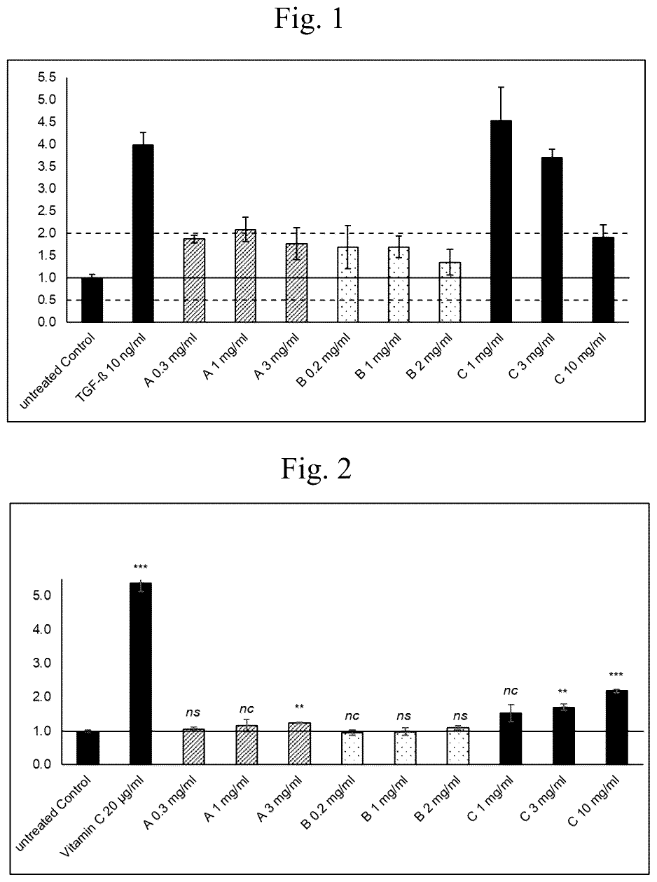

1. A cosmetic composition comprising a recombinant bacterial collagen-like protein, wherein the recombinant bacterial collagen-like protein has an amino acid sequence which is at least 60% identical to the amino acid sequence of SEQ ID NO:1 and the amino acid sequence comprises a deletion of at least 38 amino acids at the N-terminus of the amino acid sequence of SEQ ID NO:1.

2. The cosmetic composition according to claim 1, wherein the recombinant bacterial collagen-like protein is a collagen-like protein from Streptococcus pyogenes.

3. The cosmetic composition according to claim 1, wherein the recombinant bacterial collagen-like protein is obtained by fermentation in a host cell and the host cell is a yeast cell or a bacterial cell.

4. The cosmetic composition according to claim 1, wherein the amino acid sequence of the recombinant bacterial collagen-like protein comprises a deletion of between 38 and 90 amino acids at the N-terminus of the amino acid sequence of SEQ ID NO:1.

5. The cosmetic composition according to claim 1, wherein the amino acid sequence of the recombinant bacterial collagen-like protein is at least 60% identical to the amino acid sequence of SEQ ID NO: 2, SEQ ID NO:3, SEQ ID NO:4, SEQ ID NO:5, SEQ ID NO:6, SEQ ID NO:7, SEQ ID NO:8 or SEQ ID NO:9.

6. The cosmetic composition according to claim 1, wherein the amino acid sequence of the recombinant bacterial collagen-like protein is at least 90%, 92%, 94%, 96%, 97%, 98%, 99% or 100% identical to the amino acid sequence of SEQ ID NO:2, SEQ ID NO:3, SEQ ID NO:4, SEQ ID NO:5, SEQ ID NO:6, SEQ ID NO:7, SEQ ID NO: 8 or SEQ ID NO:9.

7. The cosmetic composition according to claim 1, wherein the amino acid sequence of the recombinant bacterial collagen-like protein is 100%, identical to the amino acid sequence of SEQ ID NO:2, SEQ ID NO:3, SEQ ID NO:4, SEQ ID NO:5, SEQ ID NO:6, SEQ ID NO: 7, SEQ ID NO: 8 or SEQ ID NO:9.

8. The cosmetic composition according to claim 1, further comprising at least one additional ingredient selected from the group consisting of an emollient, an emulsifier, a thickener/viscosity regulator, a UV light protection filter, an antioxidant, a hydrotrope, a solid and filler, a film former, an anticaking agent, a pearlescence additive, a deodorant and antiperspirant active ingredient, an insect repellent, a self-tanning agent, a preservative, a conditioning agent, a perfume, a dye, an odour absorber, a superfatting agent, a carrier material, an additional skin active and a solvent.

9. A topical formulation comprising a recombinant bacterial collagen-like protein.

10. The topical formulation according to claim 9, further comprising at least one cosmetically acceptable carrier.

11. The topical formulation according to claim 10, wherein the at least one cosmetically acceptable carrier is an aqueous carrier in a single phase, biphase or emulsion.

12. An oral formulation comprising a recombinant bacterial collagen-like protein.

13. The oral formulation according to claim 12, further comprising at least one ingredient selected from a carrier, a preservative, and/or an additional edible ingredient .

14. A method of using a recombinant bacterial collagen-like protein in cosmetic applications, the method comprising:
applying a cosmetic composition comprising the recombinant bacterial collagen-like protein to skin or hair.

15. A method of using a recombinant bacterial collagen-like protein as a nutricosmetic, the method comprising:
orally administering a formulation comprising the recombinant bacterial collagen-like protein.

16. The cosmetic composition according to claim 1, wherein the host cell is Pichia pastoris.

17. The cosmetic composition according to claim 1, wherein the host cell is E. coli.

18. The cosmetic composition according to claim 1, wherein the host cell is Brevibacillus choshinensis or Corynebacterium glutamicum.

19. The cosmetic composition according to claim 1, wherein the amino acid sequence of the recombinant bacterial collagen-like protein is at least 97% identical to the amino acid sequence of SEQ ID NO:2, SEQ ID NO:3, SEQ ID NO:4, SEQ ID NO: 5, SEQ ID NO:6, SEQ ID NO:7, SEQ ID NO:8 or SEQ ID NO:9.

20. The cosmetic composition according to claim 1, wherein the amino acid sequence of the recombinant bacterial collagen-like protein is at least 98% identical to the amino acid sequence of SEQ ID NO:2, SEQ ID NO:3, SEQ ID NO:4, SEQ ID NO: 5, SEQ ID NO:6, SEQ ID NO:7, SEQ ID NO:8 or SEQ ID NO:9.


SEQUENCE LISTING (9 sequences)

SEQ ID NO: 1 - protein, 318 aa, Streptococcus pyogenes
ADEQEEKAKVRTELIQELAQGLGGIEKKNFPTLGDEDLDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 2 - protein, 280 aa, Streptococcus pyogenes
DHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 3 - protein, 255 aa, Streptococcus pyogenes
LLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 4 - protein, 240 aa, Streptococcus pyogenes
GSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 5 - protein, 255 aa, Streptococcus pyogenes
LLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPAGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 6 - protein, 255 aa, Streptococcus pyogenes
LLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPQGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 7 - protein, 255 aa, Streptococcus pyogenes
LLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPAGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 8 - protein, 255 aa, Streptococcus pyogenes
LLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPSGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 9 - protein, 255 aa, Streptococcus pyogenes
LLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPKGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY